(19) United States
(12) Patent Application Publication
(10) Pub. No.: US 2013/0123517 A1
(43) Pub. Date: May 16, 2013

(54) SILVER VANADIUM PHOSPHATES

(71) Applicants: Andrey Karpov, Metuchen, NJ (US); Cornelia Katharina Dobner, Ludwigshafen (DE); Frank Rosowski, Berlin (DE); Patrick Hubach, Hassloch (DE); Robert Glaum, Rheinbach-Flerzheim (DE); Stephan Schunk, Heidelberg-Rohrbach (DE); Gerhard Cox, Bad Durkheim (DE)

(72) Inventors: Andrey Karpov, Metuchen, NJ (US); Cornelia Katharina Dobner, Ludwigshafen (DE); Frank Rosowski, Berlin (DE); Patrick Hubach, Hassloch (DE); Robert Glaum, Rheinbach-Flerzheim (DE); Stephan Schunk, Heidelberg-Rohrbach (DE); Gerhard Cox, Bad Durkheim (DE)

(73) Assignee: BASF SE, Ludwigshafen (DE)

(21) Appl. No.: 13/672,855

(22) Filed: Nov. 9, 2012

Related U.S. Application Data
(60) Provisional application No. 61/558,460, filed on Nov. 11, 2011.

(51) Int. Cl. B01J 27/198; C07D 307/60

(52) U.S. Cl. CPC B01J 27/198; C07D 307/60
USPC 549/262; 502/209

(57) ABSTRACT

The invention relates to novel silver vanadium phosphates, catalysts based on these silver vanadium phosphates and the use of these catalysts for carrying out organic reactions in the gas phase.

The invention relates to novel silver vanadium phosphates, catalysts based on these silver vanadium phosphates and the use of these catalysts for carrying out organic reactions in the gas phase.

Vanadium phosphates display a great structural variety which is associated with interesting property profiles. Thus, vanadium phosphates and vanadium phosphate-comprising metal oxides are used, inter alia, as heterogeneous catalysts for organic reactions or as ion exchangers. Numerous vanadium phosphates have a layer structure and allow, for example, the preparation of intercalation compounds having unusual magnetic properties.

Silver-comprising vanadium phosphates have recently attracted a great deal of attention since they could be suitable for the construction of high-performance batteries. Thus, A. Grandin et al. in J. Solid State Chem., vol. 115 (1995), pages 521 to 524, describe a silver vanadium(III) phosphate having the composition AgV₂P₃O₁₁. It is prepared by reacting AgNO₃, (NH₄)₂HPO₄, V₂O₅ and WO₃ at 380° C. and subsequently at 950° C. Attempts to prepare the compound in phase-pure form were unsuccessful.

A silver vanadium(IV) phosphate having the composition Ag₂VP₂O₈ is disclosed by A. Daidouh et al. in J. Solid State Chem., vol. 130 (1997), pages 28 to 34. It is prepared by reacting AgNO₃, (NH₄)₂HPO₄ and V₂O₄ at 400° C., 500° C. and subsequently at 550° C.

A silver vanadium(IV, V) phosphate having the composition AgV₂P₂O₁₀ is described by A. Grandin et al. in J. Solid State Chem., vol. 104 (1993), pages 226 to 231. It was prepared by reacting AgNO₃, (NH₄)₂HPO₄ and V₂O₅ in a ratio of 10/20/9 (atomic ratio) at 380° C., subsequently adding vanadium to an atom ratio of Ag/P/V=10/20/20 and heating the mixture at 900° C. for 24 hours. Single crystals of AgV₂P₂O₁₀ could be isolated from the resulting mixture. Attempts to prepare the compound in phase-pure form were unsuccessful.

Further silver vanadium phosphates are described by H.-Y. Kang et al. in J. Chem. Soc., Dalton Trans., (1993), pages 1525 to 1528 (silver vanadium(V) phosphate Ag₂VPO₆), P. Ayyappan et al. in Inorg. Chem., vol. 37 (1998), pages 3628 to 3634 (silver vanadium (IV, V) phosphate dihydrate Ag_{0.43}VPO₅×2H₂O), M. Asnani et al. in Eur. J. Inorg. Chem., (2005), pages 401 to 409 (silver vanadium(V) phosphate Ag_{3.5}VP_{1.5}O₈), Y. J. Kim et al. in J. Power Sources, vol. 196 (2011), pages 3325 to 3330 (Ag_{0.48}VPO₅×1.9H₂O) and J. Liu et al. in Chem. Eng. J., vol. 151 (2009), pages 319 to 323 (catalysts based on silver-doped vanadium phosphates having atomic ratios of Ag/V=0.05, 0.1 and 0.15 and comprising small amounts of Ag₂VO₂PO₄ and AgV₂P₂O₁₀ and optionally (VO)₂P₂O₇ in addition to the main phase β-VOPO₄ and their use in the liquid-phase oxidation of styrene).

In Chem. Ing. Tech., Vol. 83 (2011), pages 1697 to 1704, A Karpov et al. describe catalysts based on silver vanadium phosphates having atomic ratios of Ag/V/P=2/1/1, 2/1/2 and 2/1/1.6 and their use in the gas-phase oxidation of n-butane.

It was an object of the invention to provide novel silver vanadium phosphates and a process for preparing them. A further object of the invention was to develop processes for using these silver vanadium phosphates as heterogeneous catalysts for chemical reactions.

The invention accordingly provides novel silver vanadium phosphates of the general formula (I)

AgₐV₁P_{b}M_{c}O_{d} (I)
where
M is at least one element selected from the group consisting of H, Li, Na, K, Rb, Cs, Mg, Ca, Al, Ga, Si, Nb, Co, Cu and Zn,
a is from 0.7 to 1.3,
b is from 0.8 to 2.0,
c is less than 0.25 and
d is from 3.85 to 8.375 and indicates the number of O²⁻ ions in the formula (I) which are required to achieve electric neutraility given the oxidation state and abundance of the elements other than oxygen.

A preferred embodiment of the invention provides silver vanadium phosphates of the general formula (I) in which M is at least one element selected from the group consisting of H, Li, Na, K, Rb, Cs, Mg, Ca, Co, Cu and Zn and particularly preferably consisting of H, Li, Na, K, Rb and Cs.

A further preferred embodiment of the invention provides silver vanadium phosphates of the general formula (I) in which a is from 0.75 to 1.1.

A further preferred embodiment of the invention provides silver vanadium phosphates of the general formula (I) in which b is from 0.8 to 1.5.

A further preferred embodiment of the invention provides silver vanadium phosphates of the general formula (I) in which c is less than 0.1, particularly preferably 0.

A further preferred embodiment of the invention provides silver vanadium phosphates of the general formula (I) in which d is from 4.85 to 6.5.

A further preferred embodiment of the invention provides silver vanadium phosphates of the general formula (I) in which the average vanadium oxidation state (determined by potentiometric titration) is in the range from +3.7 to +4.7, particularly preferably from +3.9 to +4.4.

In a preferred embodiment of the invention, the silver vanadium phosphates of the general formula (I) have an X-ray powder diffraction pattern having at least three reflections at lattice plane spacings d selected from the group consisting of d=3.15±0.04, 2.74±0.15, 2.23±0.04, 2.06±0.04, 2.00±0.04, 1.79±0.04, 1.58±0.04, 1.49±0.04 and 1.42±0.04 angstoms (Å). In a particularly preferred embodiment of the invention, the silver vanadium phosphates of the general formula (I) have an X-ray powder diffraction pattern having a reflection at a lattice plane spacing d=3.15±0.04 angstoms (Å) and at least 3 reflections at lattice plane spacings d selected from the group consisting of d=2.74±0.15, 2.23±0.04, 2.06±0.04, 2.00±0.04, 1.79±0.04, 1.58±0.04, 1.49±0.04 and 1.42±0.04 angstoms (Å).

In a further preferred embodiment of the invention, the silver vanadium phosphates of the general formula (I) have a BET surface area of at least 1 m²/g, preferably at least 3 m²/g and particularly preferably at least 5 m²/g.

The invention further provides a process for preparing silver vanadium phosphates of the general formula (I), which comprises the steps
(i) reaction of at least one vanadium(V) compound with a reducing agent in a solvent,
(ii) reaction of the reaction mixture from step (i) with at least one silver compound and at least one phosphorus compound and optionally a compound of an element M,
(iii) removal of the solvent and isolation of the solid,
(iv) thermal treatment of the solid under a controlled atmosphere.

In step (i) of the process, at least one vanadium(V) compound is reacted with a reducing agent in a solvent. Possible vanadium(V) compounds are, for example, vanadium pentoxide, ammonium metavanadate, vanadyl trichloride, vanadium(V) oxytriethoxide and vanadium(V) oxytriisopropoxide.

In a preferred embodiment of the invention, the at least one vanadium(V) compound is used in admixture with at least one vanadium(IV) compound. Possible vanadium(IV) compounds are, for example, vanadium(IV) oxide, vanadium(IV) chloride and vanadyl sulfate.

As reducing agent, it is possible to use, for example, organic acids (e.g. citric acid, malonic acid), alcohols (e.g. ethanol, propanol, isobutanol, benzyl alcohol) or hydrogen peroxide, hydrazine or hydroxylamine.

A possible solvent is first and foremost water. However, it is also possible to use mixtures of water with organic solvents such as alcohols, ketones, esters or the like. The solvent used can simultaneously also serve as reducing agent, e.g. in the case of alcohols.

Both the vanadium(V) compound or the mixture of the vanadium(V) compound and the vanadium(IV) compound and also the reducing agent can be entirely or partially insoluble in the solvent used. The reaction can therefore be carried out either in homogeneous solution or in heterogeneous suspension.

The stoichiometric ratio of the vanadium(V) compound and the reducing agent is generally in the range from 0.05 to 10, preferably in the range from 0.2 b is 1.

The reaction is generally carried out at a temperature in the range from 0 to 220° C., preferably in the range from 40 to 120° C., and for a time of from 0.5 to 48 hours. If necessary, the reaction can be carried out under superatmospheric pressure, preferably in the range from atmospheric pressure to 10 bar.

In step (ii) of the process of the invention, at least one silver compound and at least one phosphorus compound and optionally a compound of an element M are added to the product from step (i). As silver compound, it is possible to use, for example, silver oxide, silver acetate, silver nitrate or silver chloride, preferably silver oxide or silver acetate. As phosphorus compound, it is possible to use, for example, phosphoric acid, phosphorus pentoxide, ammonium dihydrogenphosphate, diammonium hydrogenphosphate or alkali metal and alkaline earth metal phosphates. As compounds of the element M, it is possible to use, for example, M oxides, M acetates, M nitrates or M chlorides.

The ratios depend on the desired composition of the product. In step (ii), too, the reaction mixture can be present as solution or suspension. The reaction in step (ii) is generally likewise carried out at a temperature in the range from 0 to 220° C., preferably in the range from 40 to 120° C., and for a time of from 0.5 to 48 hours.

In step (iii) of the process of the invention, the solvent is separated off and the solid is isolated. The solid can be separated off from a suspension by, for example, filtration, centrifugation or another operation with which those skilled in the art will be familiar. Part of the solvent can possibly be evaporated beforehand. However, the solvent can also be evaporated completely, for instance in the case of spray drying.

The solid isolated in step (iii) generally has an average oxidation state of vanadium of +3.7 to +4.7 and preferably from +3.9 to +4.4.

The solid obtained is finally subjected to thermal treatment under a controlled atmosphere in step (iv) of the process of the invention.

In a preferred embodiment of the invention, the thermal treatment comprises the following steps:
(iv1) Heating of the solid in an oxidizing atmosphere having an oxygen content in the range from 2 to 21% by volume at temperatures in the range from 200 to 350° C. for from 0.1 to 24 hours and
(iv2) Heating of the solid in a nonoxidizing atmosphere having an oxygen content of ≦0.5% by volume at temperatures in the range from 300 to 600° C. for a time of ≧0.5 hours.

Step (iv1) is generally carried out after a heating-up phase.

In a preferred embodiment of the invention, air or a mixture of air with inert gases (e.g. nitrogen or argon) and/or steam is used in step (iv1) of the thermal treatment. The temperature can be kept constant, increase or decrease during step (iv1). The time of the thermal treatment in step (iv1) is preferably selected so that an average oxidation state of vanadium of from +3.7 to +4.7, preferably from +3.9 to +4.4, is established. The time required in step (iv1) is generally dependent on the nature of the reducing agent used in step (i) and the V, Ag, P and M compounds used in steps (i) and (ii), on the temperature set in step (iv1) and on the gas atmosphere selected, in particular the oxygen content. In general, the thermal treatment in step (iv1) is carried out for a time in the range from 0.1 to 24 hours, preferably in the range from 0.5 to 6 hours, in order to set the desired average vanadium oxidation state.

The nonoxidizing atmosphere in step (iv2) generally comprises inert gases (e.g. nitrogen or argon) and/or steam. The nonoxidizing atmosphere preferably comprises from 25 to 100% by volume of nitrogen and from 0 to 75% by volume of steam. The nonoxidizing atmosphere particularly preferably comprises from 40 to 75% by volume of nitrogen and from 25 to 60% by volume of steam. During step (iv2), too, the temperature can be kept constant, increase or decrease. In general, the thermal treatment in step (iv2) is carried out at temperatures in the range from 300 to 600° C., preferably in the range from 300 to 500° C. and particularly preferably in the range from 330 to 450° C. In general, the thermal treatment in step (iv2) is carried out for a time of more than 0.5 hours, preferably in the range from 2 to 12 hours. In step (iv2), the solid is very particularly preferably heated to a temperature of 330° C.-375° C. over a period of from 0.5 to 3 hours, maintained at this temperature for a time of up to 1 hour, then heated to a temperature of 375° C.-450° C. over a period of from 0.2 to 2 hours and maintained at this temperature for a time of from 2 to 6 hours.

After step (iv2), the solid is generally cooled in a nonoxidizing atmosphere having an oxygen content of ≦0.5% by volume to a temperature of ≦300° C., preferably ≦200° C. and particularly preferably ≦150° C.

Before the thermal treatment in step (iv), the solid can optionally be subjected to shaping and shaped to give, for example, pellets, hollow cylinders, crushed materials or extrudates. This shaping is preferably carried out by tableting, advantageously with prior mixing with a lubricant such as graphite.

The invention further provides a catalyst comprising a silver vanadium phosphate of the general formula (I) as described above.

The invention further provides for the use of silver vanadium phosphates of the general formula (I) as heterogeneous catalysts for carrying out chemical reactions.

In a preferred embodiment of the invention, the silver vanadium phosphates of the general formula (I) are used as heterogeneous catalysts for carrying out organic reactions, in particular for the partial oxidation of alkanes such as ethane, n-propane, i-propane, n-butane or i-butane, alkenes such as ethene, propene, 1-butene, i-butene, 2-isobutene, 2-trans-butene or butadiene, aromatics such as benzene or naphthalene, alkylaromatics such as toluene or xylenes, aldehydes such as acrolein or methacrolein, for the dehydration of alcohols such as glycerol or for the reaction of alcohols with acids or aldehydes, for example methanol with acetic acid or ethanol with formaldehyde.

The invention is illustrated by the following examples and figures.

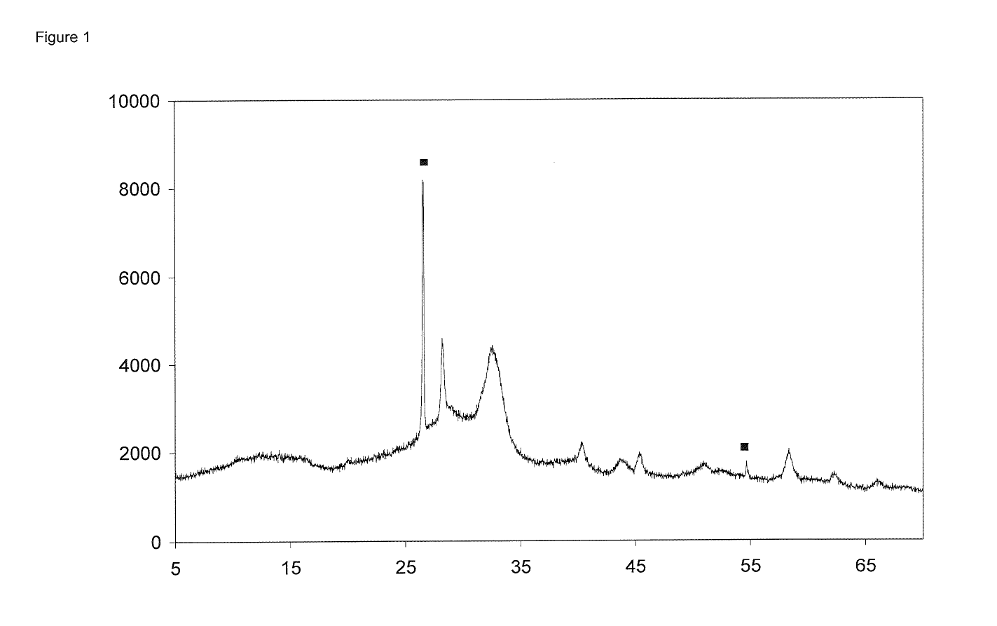

FIG. 1 shows the X-ray powder diffraction pattern of the composition Ag₁V₁P₁O_{5.065} according to the invention comprising ≦1% by weight of graphite.

All X-ray diffraction patterns were recorded using a diffractometer from Bruker AXS GmbH, 76187 Karlsruhe, instrument designation: D8 Advance with LYNXEYE detector. Cu—Kα radiation (40 kV, 40 mA) was used for recording the diffraction patterns.

The average oxidation state of vanadium was determined by potentiometric titration as described in WO 02/34387.

The BET surface area was determined on the Autosorb-6b instrument from Quantachrome in accordance with DIN 66131.

### EXAMPLE S1

### Production of a Suspension S1

1l of water was placed in a 2.5 l stirred glass vessel flushed with nitrogen. 90.94 g of V₂O₅ (from Gfe, purity 99.9%) were subsequently added over a period of 2 minutes while stirring (300 rpm) and rinsed in with 0.3 l of water. The suspension was heated to 60° C. 106.13 g of citric acid (Bernd-Kraft GmbH, purity 99%) together with 0.2 l of water were subsequently added and the mixture was stirred at 60° C. for 7 hours. 117.04 g of Ag₂O (from Lancaster, purity 99%) together with 0.25 l of water and 115.29 g of H₃PO₄ (from Bernd-Kraft GmbH, purity 85%) together with 0.25 l of water were subsequently added while stirring. The suspension was heated to 90° C. and stirred at this temperature for 16 hours.

### EXAMPLE S2

### Production of a Suspension S2

The suspension was produced in a manner analogous to example S1. Instead of 106.13 g of citric acid, only 84.90 g were used. The stirring time at 60° C. was 24 hours.

### EXAMPLE S3

### Production of a Suspension S3

The suspension was produced in a manner analgous to example S1. Instead of 115.29 g of H₃PO₄, 138.35 g were used.

### EXAMPLE I1

### Preparation of an Intermediate I1

The suspension S1 from example S1 was spray dried (spray dryer from Niro Inc., Mobile Minor 2000). The spray-dried powder had a BET surface area of 20 m²/g and an average oxidation state of vanadium of +3.97.

### EXAMPLE I2

### Preparation of an Intermediate I2

The suspension S1 was filtered with suction on a suction filter (pore size 4) and subsequently dried at 100° C. in a vacuum drying oven for 16 hours.

### EXAMPLE I3

### Preparation of an Intermediate I3

The suspension S2 from example S2 was spray dried (spray dryer from Niro Inc., Mobile Minor 2000). The spray-dried powder had a BET surface area of 23 m²/g and an average oxidation state of vanadium of +4.16.

### EXAMPLE I4

### Preparation of an Intermediate I4

The suspension S3 from example S3 was spray dried (spray dryer from Niro Inc., Mobile Minor 2000). The spray-dried powder had an average oxidation state of vanadium of +3.98.

The powders from examples I1 to I4 were each admixed with 1% by weight of graphite (Timrex T44), intensively mixed and processed on a compacting machine (from Powtec, model RCC 100*20, pressure=200 bar, screen mill 110.7, roller 2.4, screw 16) to give crushed material CM1 to CM4. The crushed material was sieved to produce a fraction from 0.5 to 1 mm.

### EXAMPLE 1

### Preparation of a Silver Vanadium Phosphate Ag₁V₁P₁O_{5.065}

20 g of the crushed material CM1 were introduced into an electrically heated tube, heated in a mixture of 50% by volume of nitrogen (12.5 standard l/h) and 50% by volume of air (12.5 standard l/h) at a heating rate of 5° C./min to 250° C. and maintained at this temperature for 50 minutes. The gas atmosphere was subsequently changed to a mixture of 50% by volume of nitrogen (12.5 standard l/h) and 50% by volume of steam (12.5 standard l/h). The crushed material was then heated at a heating rate of 1° C./min to 350° C. and maintained at this temperature for 5 minutes, then heated at a heating rate of 3° C./min to 425° C. and maintained at this temperature for 195 minutes. The gas atmosphere was subsequently changed to nitrogen (25 standard l/h) and the crushed material was cooled to room temperature.

The crushed material which had been calcined in this way had a BET surface area of 9.3 m²/g and an average oxidation state of vanadium of +4.13. Atomic emission spectroscopy indicated an Ag content of 39.5% by weight, a V content of 18.2% by weight and a P content of 10.9% by weight. This corresponds to an atomic ratio of Ag/V/P of 1/1/1.

An X-ray powder diffraction pattern was recorded on the powder obtained (FIG. 1; the abscissa shows 2-theta values in ° and the ordinate shows the associated intensity; black squares indicate the reflections of graphite). The strongest reflections in the X-ray powder diffraction pattern where found to be at the following lattice plane spacings d [Å]: 3.15±0.15, 2.74±0.15, 2.23±0.04, 2.06±0.04, 2.00±0.04, 1.79±0.04, 1.58±0.04, 1.49±0.04, 1.42±0.04. Reflections of graphite were at lattice plane spacings d [Å±0.04] of 3.35 and 1.68.

### EXAMPLE 2

### Preparation of a Silver Vanadium Phosphate Ag₁V₁P₁O_{5.095}

20 g of the crushed material CM2 were thermally treated as in example 1.

The calcined crushed material had a BET surface area of 7.1 m²/g and an average oxidation state of vanadium of +4.19. An X-ray powder diffraction pattern was recorded on the powder obtained. The strongest reflections of Ag₁V₁P₁O_{5.095} agreed with the reflections of Ag₁V₁P₁O_{5.065} from example 1.

### EXAMPLE 3

### Preparation of a Silver Vanadium Phosphate Ag₁V₁P₁O_{5.1}

20 g of the crushed material CM3 were thermally treated as in example 1.

The calcined crushed material had a BET surface area of 8 m²/g and an average oxidation state of vanadium of +4.2. An X-ray powder diffraction pattern was recorded on the powder obtained. The strongest reflections of Ag₁V₁P₁O_{5.1} agreed with the reflections of Ag₁V₁P₁O_{5.065} from example 1.

### EXAMPLE 4

### Preparation of a Silver Vanadium Phosphate Ag₁V₁P₁O_{5.085}

20 g of the crushed material CM1 were thermally treated as in example 1. However, reaching 425° C., the atmosphere was changed to 100% by volume of nitrogen (25 standard l/h), the crushed material was maintained at this temperature for 300 minutes and subsequently cooled to room temperature under 100% by volume of nitrogen (25 standard l/h).

The calcined crushed material had an average oxidation state of vanadium of +4.17. An X-ray powder diffraction pattern was recorded on the powder obtained. The strongest reflections of Ag₁V₁P₁O_{5.05} agreed with the reflections of Ag₁V₁P₁O_{5.065} from example 1.

### EXAMPLE 5

### Preparation of a Silver Vanadium Phosphate Ag₁V₁P_{1.2}O_{5.52}

20 g of the crushed material CM4 were thermally treated as in example 1.

The calcined crushed material had an average oxidation state of vanadium of +4.04.

### EXAMPLE 6

### Preparation of a Silver Vanadium Phosphate Ag₁V₁P_{1.2}O_{5.53}

20 g of the crushed material CM4 were introduced into an electrically heated tube, heated in a mixture of 50% by volume of nitrogen (12.5 standard l/h) and 50% by volume of air (12.5 standard l/h) at a heating rate of 5° C./min to 250° C. and maintained at this temperature for 50 minutes. The gas atmosphere was subsequently changed to a mixture of 42% by volume of nitrogen (12.5 standard l/h) and 42% by volume of steam (12.5 standard l/h) and 16% by volume of air (4.6 standard l/h). The crushed material was then heated at a heating rate of 1° C./min to 350° C. The gas atmosphere was subsequently changed to a mixture of 50% by volume of nitrogen (12.5 standard l/h) and 50% by volume of steam (12.5 standard l/h). The crushed material was maintained at 350° C. for 5 minutes, then heated at a heating rate of 3° C./min to 425° C. and maintained at this temperature for 195 minutes. The gas atmosphere was subsequently changed to nitrogen (25 standard l/h) and the crushed material was cooled to room temperature.

The calcined crushed material had an average oxidation state of vanadium of +4.04.

TABLE 1
Overview of the designations of the intermediate and products

| | | Crushed | |
|---|---|---|---|
| Suspension | Intermediate | material | Example |
| S1 | I1 | CM1 | 1 |
| S1 | I2 | CM2 | 2 |
| S2 | I3 | CM3 | 3 |
| S1 | I1 | CM1 | 4 |
| S3 | I4 | CM4 | 5 |
| S3 | I4 | CM4 | 6 |

Catalytic testing of the products from examples 1 to 6 was carried out for the conversion of n-butane into maleic anhydride.

Catalytic testing was in each case carried out on 1 ml of the sample in a 48-fold test reactor as described in DE 198 09 477 A1. The composition of the reaction gas mixture is defined by the concentration of n-butane, air, steam and triethyl phosphate. The balance to 100% by volume was argon. The product gas stream was analyzed by gas chromatography (GC 6890, from Agilent).

In the present text, the selectivity of maleic anhydride formation (S^{MAn} (mol %)) is:

$${S}^{MAn} = \frac{\begin{matrix}mol of n \text{-} butane converted \\ into maleic anhydride\end{matrix}}{total mol of n \text{-} butane reacted} \times 100$$

(the conversions are in each case based on a single pass of the reaction gas mixture through the fixed catalyst bed).

The conversion C^{C4} of n-butane (mol %) is defined correspondingly:

$${C}^{C 4} = \frac{mol of n \text{-} butane reacted}{mol of n \text{-} butane used} \times 100.$$

The following results were obtained in the reaction of n-butane:

TABLE 2
Results for the use of silver vanadium phosphates according to
the invention as catalysts for the oxidation of butane

| | | | C4 | Air | | | | |
|---|---|---|---|---|---|---|---|---|
| | | | [% | [% | | | | |
| | | | by | by | Water | TEP | | |
| | T | GHSV | vol- | vol- | [% by | [ppm by | C^{C4} | S^{MAn} |
| Catalyst | [° C.] | [h−1] | ume] | ume] | volume] | volume] | [%] | [%] |
| Ex. 1 | 400 | 2000 | 1.95 | 92.63 | 3 | 1 | 13 | 11 |
| Ex. 2 | 400 | 2000 | 1.95 | 92.63 | 3 | 1 | 9 | 12 |
| Ex. 3 | 400 | 2000 | 1.95 | 92.63 | 3 | 1 | 8 | 13 |
| Ex. 4 | 400 | 2000 | 1.95 | 92.63 | 3 | 1 | 7 | 19 |
| Ex. 5 | 400 | 2000 | 1.95 | 92.63 | 3 | 1 | 8 | 13 |
| Ex. 6 | 400 | 2000 | 1.95 | 92.63 | 3 | 1 | 7 | 14 |

T—reactor temperature
GHSV—gas hourly space velocity − gas volume/catalyst volume per hour
C4—concentration of n-butane in the feed gas
Air—concentration of air in the feed gas
Water—concentration of steam in the feed gas
TEP—concentration of triethyl phosphate in the feed gas

1.-11. (canceled)

12. A silver vanadium phosphate of the general formula (I)
AgₐV₁P_{b}M_{c}O_{d} (I)
where
M is at least one element selected from the group consisting of H, Li, Na, K, Rb, Cs, Mg, Ca, Al, Ga, Si, Nb, Co, Cu and Zn,
a is from 0.7 to 1.3,
b is from 0.8 to 2.0,
c is less than 0.25 and
d is from 3.85 to 8.375 and indicates the number of O²⁻ ions in the formula (I) which are required to achieve electric neutraility given the oxidation state and abundance of the elements other than oxygen.

13. The silver vanadium phosphate according to claim 12, wherein M is at least one element selected from the group consisting of H, Li, Na, K, Rb, Cs, Mg, Ca, Co, Cu and Zn.

14. The silver vanadium phosphate according to claim 12, wherein d is from 4.85 to 6.5.

15. The silver vanadium phosphate according to claim 12, wherein the average vanadium oxidation state is in the range from +3.7 to +4.7.

16. The silver vanadium phosphate according to claim 12 which has a BET surface area of at least 1 m²/g.

17. The silver vanadium phosphate according to claim 12, wherein the silver vanadium phosphate has an X-ray powder diffraction pattern having at least three reflections at lattice plane spacings d selected from the group consisting of d=3.15±0.04, 2.74±0.15, 2.23±0.04, 2.06±0.04, 2.00±0.04, 1.79±0.04, 1.58±0.04, 1.49±0.04 and 1.42±0.04 angstoms (Å).

18. The silver vanadium phosphate according to claim 12, wherein the silver vanadium phosphate has an X-ray powder diffraction pattern having a reflection at a lattice plane spacing d=3.15±0.04 angstoms (Å) and at least 3 reflections at lattice plane spacings d selected from the group consisting of d=2.74±0.15, 2.23±0.04, 2.06±0.04, 2.00±0.04, 1.79±0.04, 1.58±0.04, 1.49±0.04 and 1.42±0.04 angstoms (Å).

19. A process for preparing the silver vanadium phosphate of the general formula (I) according to claim 12, which comprises the steps
(i) reaction of at least one vanadium(V) compound with a reducing agent in a solvent,
(ii) reaction of the reaction mixture from step (i) with at least one silver compound and at least one phosphorus compound and optionally a compound of an element M,
(iii) removal of the solvent and isolation of the solid,
(iv) thermal treatment of the solid under a controlled atmosphere.

20. The process according to claim 19, wherein the thermal treatment in step (iv) comprises the following steps:
(iv1) Heating of the solid in an oxidizing atmosphere having an oxygen content in the range from 2 to 21% by volume at temperatures in the range from 200 to 350° C. for from 0.1 to 24 hours and
(iv2) Heating of the solid in a nonoxidizing atmosphere having an oxygen content of ≦0.5% by volume at temperatures in the range from 300 to 600° C. for a time of ≧0.5 hours.

21. A catalyst comprising the silver vanadium phosphate according to claim 12.

22. The use of silver vanadium phosphates of the general formula (I) according to claim 12 as heterogeneous catalysts for carrying out chemical reactions.
